(19) United States
(12) Patent Application Publication
(10) Pub. No.: US 2026/0306186 A1
(43) Pub. Date: Oct. 8, 2026

(54) NON-INVASIVE PATIENT TRACKER FOR SURGICAL PROCEDURE

(71) Applicant: Mazor Robotics Ltd., Caesarea (IL)

(72) Inventors: Weijun Xu, Shanghai (CN); Wei Tang, Shanghai (CN); Fang Geng, Shanghai (CN)

(21) Appl. No.: 19/482,428

(22) PCT Filed: May 8, 2023
(86) PCT No.: PCT/CN2023/092720

§ 371 Date: Nov. 7, 2025

(51) Int. Cl. A61B 34/20; A61B 34/30

(52) U.S. Cl. CPC A61B 34/20; A61B 34/30; A61B 2034/2051; A61B 2034/2055; A61B 2034/2065

(57) ABSTRACT

A surgical system (100), navigation system (118), and method are provided. The method includes obtaining, via a first imaging device (112a), a first image of an object in a surgical area; obtaining, via a second imaging device (112b), a second image of the first imaging device (112a ), where the second image comprises one or more second tracking markers (412) arranged in a first position. The method further includes obtaining, via the first imaging device (112a), one or more additional first images of the object; obtaining, via the second imaging device (112b), one or more additional second images of the first imaging device (112a); and determining, based on the one or more additional first images and the one or more additional second images, whether the object has moved and/or whether the first imaging device (112a) has moved relative to the object.

### FIELD

The present disclosure is generally directed to surgical systems, and relates more particularly to robotic surgical devices.

### BACKGROUND

Surgical robots may assist a surgeon or other medical provider in carrying out a surgical procedure, or may complete one or more surgical procedures autonomously. Layout of the operating room during a surgical procedure is especially important to support successful use of surgical robots.

### BRIEF SUMMARY

Example aspects of the present disclosure include:
A method, comprising: obtaining, via a first imaging device, a first image of an object in a surgical area, where the first image includes one or more first tracking markers arranged in a first position relative to the object; obtaining, via a second imaging device, a second image of the first imaging device, where the second image includes one or more second tracking markers arranged in a first position; obtaining, via the first imaging device, one or more additional first images of the object, where the one or more additional first images includes the one or more first tracking markers; obtaining, via the second imaging device, one or more additional second images of the first imaging device, where the one or more additional second images include the one or more second tracking markers; and determining, based on the one or more additional first images and the one or more additional second images, whether the object has moved and/or whether the first imaging device has moved relative to the object.

In some aspects, the object comprises soft tissue, and the method further includes: determining, based on the one or more additional first images, that the object has moved beyond a predetermined object motion threshold; and in response to determining that object has moved beyond the predetermined object motion threshold, reporting soft tissue motion to at least one of a surgeon and a robot.

In some aspects, the soft tissue motion is reported to the robot, and the method further includes: adjusting a robotic surgical plan based on the soft tissue motion.

In some aspects, the first imaging device is connected to the robot.

In some aspects, the first imaging device is connected to at least one of a robotic arm and an end effector of the robot.

In some aspects, the method further includes: determining, based on the one or more additional second images, that the first image device has moved relative to the object beyond a predetermined distance threshold; correlating motion of the first imaging device to motion of the robot; and in response to determining that the first image device has moved relative to the object beyond a predetermined distance threshold, reporting robot or patient movement to the surgeon.

In some aspects, the one or more first tracking markers are provided on a flexible material and the flexible material is attached to a patient at or near a surgical area.

In some aspects, the flexible material is attached to the patient using at least one of a staple, suture, and glue.

In some aspects, wherein the one or more first tracking markers are sized according to a resolution of the first imaging device and are not visible to the second imaging device.

In some aspects, the one or more first tracking markers are printed on the flexible material.

In some aspects, the flexible material includes a center area that is substantially devoid of the one or more first tracking markers and the one or more first tracking markers are disposed radially around the center area.

In some aspects, the center area includes an area through which an incision is made.

In some aspects, the flexible material includes a transparent or semitransparent material.

In some aspects, the one or more second tracking markers are larger than the one or more first tracking markers and wherein the one or more second tracking markers are attached to the first imaging device.

In some aspects, the method further includes: determining, based on the one or more additional first images, that the one or more first tracking markers have moved to a second position relative to the object; determining, based on the one or more additional second images, that the one or more second tracking markers have moved to a second position indicating a motion of the first imaging device relative to the object; and reporting motion of the object and motion of the first imaging device.

In some aspects, at least one of the first image and the second image include one or more of an ultrasound image, a magnetic resonance image, a fluoroscopic image, an infrared image, a visible light image, a radiation image, a computed tomography image, a nuclear medicine image, and a positron-emission tomography image.

Another aspect includes a system, where the system includes: a first imaging device including a first field of view that captures one or more first tracking markers arranged on or around a surgical area; a second imaging device including a second field of view that captures the first imaging device; and a computing device, where the computing device includes: a processor; and computer memory coupled with the processor and having data stored therein that, when executed by the processor, enables the processor to: receive first image data from the first imaging device, where the first image data indicates a motion of the one or more first tracking markers relative to the surgical area; receive second image data from the second imaging device, where the second image data indicates a motion of the first imaging device relative to the surgical area; and determine, based on a combination of the first image data and the second image data, whether an object in the surgical area has moved and/or whether the first imaging device has moved relative to the object.

In some aspects, one or more second tracking markers are attached to the first imaging device, where the one or more first tracking markers are smaller than the one or more second tracking markers, where the second field of view includes the surgical area, and where the one or more first tracking markers are too small to be recognized in the second image data.

In some aspects, the first imaging device is connected to a robot that is supporting a surgical procedure at the surgical area and the first imaging device includes a 3D camera.

Another aspect includes a surgical system that includes: a robot configured to execute a surgical plan; a first imaging device attached to the robot and configured to capture images of one or more first tracking markers arranged on or around a surgical area, wherein the first imaging device comprises one or more second tracking markers attached thereto; a second imaging device configured to capture images of the first imaging device and the one or more second tracking markers attached thereto; and a computing device including: a processor; and computer memory coupled with the processor and having data stored therein that, when executed by the processor, enables the processor to: receive first image data from the first imaging device, where the first image data indicates a motion of the one or more first tracking markers relative to the surgical area; receive second image data from the second imaging device, where the second image data indicates a motion of the first imaging device relative to the surgical area; determine, based on a combination of the first image data and the second image data, that an object in the surgical area has moved and/or the first imaging device has moved relative to the object; and in response to determining that the object in the surgical area has moved and/or the first imaging device has moved relative to the object, update the surgical plan.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/implementations in combination with any one or more other aspects/features/implementations.

Use of any one or more of the aspects or features as disclosed herein.

It is to be appreciated that any feature described herein can be claimed in combination with any other feature(s) as described herein, regardless of whether the features come from the same described implementation.

The details of one or more aspects of the disclosure are set forth in the accompanying drawings and the description below. Other features, objects, and advantages of the techniques described in this disclosure will be apparent from the description and drawings, and from the claims.

The preceding is a simplified summary of the disclosure to provide an understanding of some aspects of the disclosure. This summary is neither an extensive nor exhaustive overview of the disclosure and its various aspects, implementations, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, implementations, and configurations of the disclosure are possible utilizing, alone or in combination, one or more of the features set forth above or described in detail below.

Numerous additional features and advantages of the present disclosure will become apparent to those skilled in the art upon consideration of the implementation descriptions provided hereinbelow.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, implementations, and configurations of the disclosure, as illustrated by the drawings referenced below.

FIG. 1 is a block diagram of a system according to at least one implementation of the present disclosure;

FIG. 2 illustrates a non-invasive patient tracker according to at least one implementation of the present disclosure;

FIG. 3A illustrates a first configuration of a flexible material with respect to a patient according to at least one embodiment of the present disclosure;

FIG. 3B illustrates a second configuration of a patient having more than one patches of flexible material applied thereto according to at least one embodiment of the present disclosure;

FIG. 4 is a plan view of an environment in which a surgical robot may operate according to at least one implementation of the present disclosure;

FIG. 5 is an example of a first process flow according to at least one implementation of the present disclosure; and

FIG. 6 is an example of a second process flow according to at least one implementation of the present disclosure.

### DETAILED DESCRIPTION

It should be understood that various aspects disclosed herein may be combined in different combinations than the combinations specifically presented in the description and accompanying drawings. It should also be understood that, depending on the example or implementation, certain acts or events of any of the processes or methods described herein may be performed in a different sequence, and/or may be added, merged, or left out altogether (e.g., all described acts or events may not be necessary to carry out the disclosed techniques according to different implementations of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10X Fusion processors; Apple A11, A12, A12X, A12Z, or A13 Bionic processors; or any other general purpose microprocessors), graphics processing units (e.g., Nvidia Geforce RTX 2000-series processors, Nvidia Geforce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any implementations of the disclosure are explained in detail, it is to be understood that the disclosure is not limited in its application to the details of construction and the arrangement of components set forth in the following description or illustrated in the drawings. The disclosure is capable of other implementations and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The terms proximal and distal are used in this disclosure with their conventional medical meanings, proximal being closer to the operator or user of the system, and further from the region of surgical interest in or on the patient, and distal being closer to the region of surgical interest in or on the patient, and further from the operator or user of the system.

Some robotic surgical system implementations include operating a robotic system within proximity of a surgical table (e.g., a surgical bed, an operating table, etc.) and a patient. The term “robotic system” may also be referred to as a “robotic surgical system” herein.

In some robotic system implementations (e.g., in which the robotic system or a robotic arm is not table-mounted or patient-mounted), movement of the robotic system relative to the surgical table and/or patient during a surgical operation may become necessary or desirable. Improved robot performance can be achieved when the robotic system and its components are tracked more accurately, especially relative to a surgical area. Aspects of the present disclosure support improving positioning and tracking of the robotic system relative to the surgical table, the patient, or a surgical area.

In some embodiments, navigation components (e.g., optical trackers or tracking markers) may be mounted at or near the surgical area, on the robot end effector, on the robot arm, on a camera, or combinations thereof. A navigation system may be provided with an ability to determine a position of the robot, the camera mounted on the robot, the robot end effector, and the robot arm relative to the surgical area by tracking a position of the navigation components. The camera mounted to the robot may further track motion of the tracking markers placed at or near the surgical area and the camera may provide images of the surgical area to a navigation system. The navigation system may utilize its view of the tracking markers and objects in the operating room along with information received from the robot-mounted camera to control the robotic surgical procedure. In particular, based on determined relative positions of the robot, the camera, the robot end effector, the robot arm, and the surgical area, the navigation system may also determine if updates to a surgical plan should be made, if the robot should be maneuvered in a particular way, etc.

Embodiments of the present disclosure provide technical solutions to one or more of the problems of (1) tracking soft tissue changes intraoperatively, (2) tracking soft tissue changes postoperatively, (3) reporting on soft tissue movement in real-time or within a clinically meaningful amount of time from soft tissue movement, and/or (4) controlling robotic navigation in response to soft tissue movement.

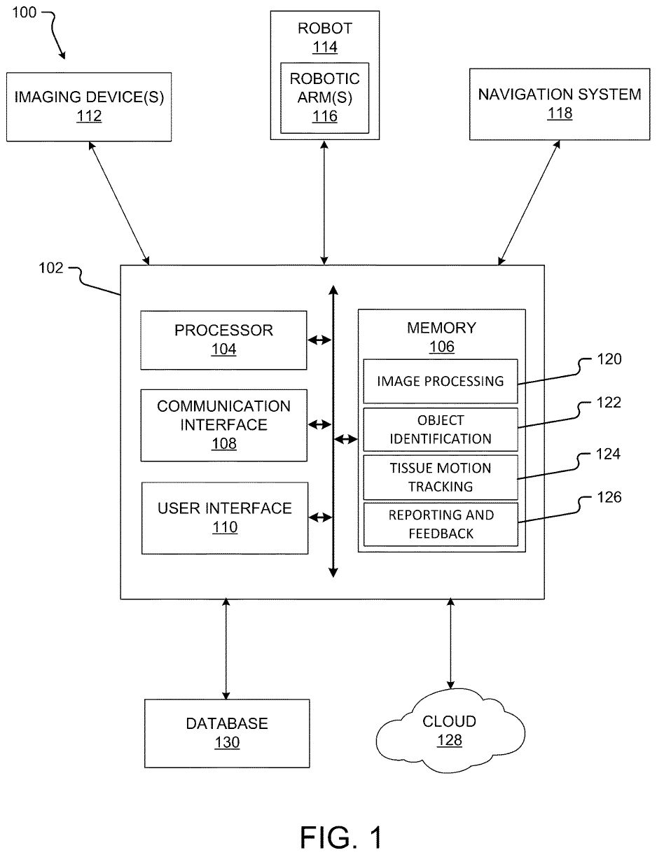

Turning first to FIG. 1, a block diagram of a system 100 according to at least one embodiment of the present disclosure is shown. The system 100 may be used to track soft tissue movement, assist robotic navigation in response to soft tissue movement, report on soft tissue movement, and/or carry out one or more other aspects of one or more of the methods disclosed herein. The system 100 illustratively includes a computing device 102, one or more imaging devices 112, a robot 114, a navigation system 118, a database 130, and/or a cloud or other network 128. Systems according to other embodiments of the present disclosure may comprise more or fewer components than the system 100. For example, the system 100 may not include the imaging device 112, the robot 114, the navigation system 118, one or more components of the computing device 102, the database 130, and/or the cloud 128.

The computing device 102, in some embodiments, comprises a processor 104, a memory 106, a communication interface 108, and a user interface 110. Computing devices according to other embodiments of the present disclosure may comprise more or fewer components than the computing device 102.

The processor 104 of the computing device 102 may be any processor described herein, a collection of processors, or any similar processor. The processor 104 may be configured to execute instructions or neural networks stored in the memory 106 (e.g., data), which may cause the processor 104 to carry out one or more computing steps utilizing or based on data received from the imaging device 112, the robot 114, the navigation system 118, the database 130, and/or the cloud 128.

The memory 106 may be or comprise RAM, DRAM, SDRAM, other solid-state memory, any memory described herein, or any other tangible, non-transitory memory for storing computer-readable data and/or instructions. The memory 106 may store information or data useful for completing, for example, any step of the methods 400, 500, 600, 700, and 800 described herein, or of any other methods. The memory 106 may store, for example, one or more image processing algorithms or neural networks 120, an object identification process 122, a tissue motion tracking process 124, and a reporting and feedback process 126. Such instructions, data, or algorithms may, in some embodiments, be organized into one or more applications, modules, packages, layers, or engines. The algorithms, data, and/or instructions may cause the processor 104 to manipulate data stored in the memory 106 and/or received from or via the imaging device 112, the robot 114, the database 130, and/or the cloud 128.

Illustratively, the memory 106 is shown to include image processing instructions 120, object identification instructions 122, tissue motion tracking instructions 124, and reporting/feedback instructions 126. Again, one or more of the instructions may be implemented as a neural network, artificial neural network, or machine learning model without departing from the scope of the present disclosure.

The image processing 120, when executed by the processor 104, may enable the computing device 102 to cooperate with the imaging device(s) 112, robot 114, and/or navigation system 118 to obtain and use patient images. In some embodiments, the image processing 120 may be configured to receive patient images (e.g., preoperative patient images, intraoperative patient images, and/or postoperative patient images), receive object images, receive environmental images (e.g., images of a surgical room) and prepare the images for processing by other components of the computing device 102. Illustratively, the image processing 120 may be configured to receive images and format such images for consumption by the object identification 122 and/or tissue motion tracking 124. In some embodiments, the image processing 120 may be configured to transform an image or image data into a different consumable format by converting the image from one digital format to another digital format, by performing pixel analysis to determine locations of objects, by identifying locations of fiducials in an image, by compressing an image, by decompressing an image, by overlaying object models on an image, and/or any other task associated with preparing an image for consumption by the object identification 122 and/or tissue motion tracking 124.

The object identification 122, when executed by the processor 104, may enable the computing device 102 to identify one or more objects in an image, identify a pose (e.g., position and/or orientation) of objects in an image and in space, identify a pose of an object relative to another object, identify a pose of objects by analyzing locations of one or more fiducials, identify patient anatomy from pixel and/or gradient analysis of images, identify a pose of non-patient objects (e.g., a robotic arm 116) relative to a patient object (e.g., an organ, nerve, muscle, bone, etc.), label objects within an image, and any other combination of tasks associated with identifying an object, locating an object, and/or differentiating one object from another object.

The tissue motion tracking 124, when executed by the processor 104, may enable the computing device 102 to track a motion of one or more objects within a field of view of an imaging device 112. In some embodiments, the tissue motion tracking 124 may be configured to identify one or more objects of interest and track a motion of the object(s) of interest intraoperatively and/or postoperatively. The tissue motion tracking 124 may be configured to track an absolute motion of one or more objects (e.g., by identifying a movement of the object in space or relative to a fixed coordinate system) and/or track a motion of one object relative to another object. The tissue motion tracking 124 may be configured to locate and track object(s) by identifying object(s) in an image via pixel analysis and then monitoring a movement of the object(s) based on an analysis of subsequent images. The tissue motion tracking 124 may be configured to track a position and/or motion of tissue (e.g., soft tissue) by identifying and tracking a location of fiducials in one or more images. As will be described herein, if a positional relationship is established between an object and one or more fiducials, then the tissue motion tracking 124 may be configured to correlate a motion of one or more fiducials to a motion of one or more objects, where the objects may include soft tissue objects such as an organ, nerve, muscle, tumor growth, skin, etc.

The reporting and feedback 126, when executed by the processor 104, may enable the computing device 102 to provide information related to object identification and/or object motion to a user of the computing device 102. Alternatively or additionally, the reporting and feedback 126 may also be configured to provide feedback to the navigation system 118 to assist with the navigation of the robot 114. As an example, the reporting and feedback 126 may provide the navigation system 118 and/or a surgeon with information related to object motion, soft tissue motion, fiducial motion, or combinations thereof. The reporting and feedback 126 may alternatively or additionally provide one or more alerts if an object or multiple objects are determined to have moved (absolutely or relatively) beyond a predetermined movement threshold. In the event that motion of an object or multiple objects exceeds the predetermined movement threshold (e.g., a predetermined distance) or two objects move too close to one another, the reporting and feedback 126 may issue an alert or cause the navigation system 118 to update a navigation plan for the robot 114. Updated navigation plans prepared in response to detecting soft tissue movement may allow the robot 114 to avoid cutting, puncturing, or otherwise damaging an object comprising soft tissue during a surgical procedure.

The computing device 102 may also comprise a communication interface 108. The communication interface 108 may be used for receiving image data or other information from an external source (such as the imaging device 112, the robot 114, the navigation system 118, the database 130, the cloud 128, and/or any other system or component not part of the system 100), and/or for transmitting instructions, images, or other information to an external system or device (e.g., another computing device 102, the imaging device 112, the robot 114, the navigation system 118, the database 130, the cloud 128, and/or any other system or component not part of the system 100). The communication interface 108 may comprise one or more wired interfaces (e.g., a USB port, an ethernet port, a Firewire port) and/or one or more wireless transceivers or interfaces (configured, for example, to transmit and/or receive information via one or more wireless communication protocols such as 802.11a/b/g/n, Bluetooth, NFC, ZigBee, and so forth). In some embodiments, the communication interface 108 may be useful for enabling the device 102 to communicate with one or more other processors 104 or computing devices 102, whether to reduce the time needed to accomplish a computing-intensive task or for any other reason.

The computing device 102 may also comprise one or more user interfaces 110. The user interface 110 may be or comprise a keyboard, mouse, trackball, monitor, television, screen, touchscreen, and/or any other device for receiving information from a user and/or for providing information to a user. The user interface 110 may be used, for example, to receive a user selection or other user input regarding any step of any method described herein. Notwithstanding the foregoing, any required input for any step of any method described herein may be generated automatically by the system 100 (e.g., by the processor 104 or another component of the system 100) or received by the system 100 from a source external to the system 100. In some embodiments, the user interface 110 may be useful to allow a surgeon or other user to modify instructions to be executed by the processor 104 according to one or more embodiments of the present disclosure, and/or to modify or adjust a setting of other information displayed on the user interface 110 or corresponding thereto.

Although the user interface 110 is shown as part of the computing device 102, in some embodiments, the computing device 102 may utilize a user interface 110 that is housed separately from one or more remaining components of the computing device 102. In some embodiments, the user interface 110 may be located proximate one or more other components of the computing device 102, while in other embodiments, the user interface 110 may be located remotely from one or more other components of the computer device 102. Illustratively, the user interface 110 may include a user input device, a user output device, and a combination user input/user output device (e.g., a touch-sensitive display device).

The imaging device 112 may be operable to image anatomical feature(s) (e.g., a bone, veins, soft tissue, etc.) and/or other aspects of patient anatomy to yield image data (e.g., image data depicting or corresponding to a bone, veins, soft tissue, etc.). “Image data” as used herein refers to the data generated or captured by an imaging device 112, including in a machine-readable form, a graphical/visual form, and in any other form. In various examples, the image data may comprise data corresponding to an anatomical feature of a patient, or to a portion thereof. The image data may be or comprise a preoperative image, an intraoperative image, a postoperative image, or an image taken independently of any surgical procedure. In some embodiments, a first imaging device 112 may be used to obtain first image data (e.g., a first image) at a first time, and a second imaging device 112 may be used to obtain second image data (e.g., a second image) at a second time after the first time. The imaging device 112 may be capable of taking a 2D image or a 3D image to yield the image data. The imaging device 112 may be or comprise, for example, an ultrasound scanner (which may comprise, for example, a physically separate transducer and receiver, or a single ultrasound transceiver), an O-arm, a C-arm, a G-arm, or any other device utilizing X-ray-based imaging (e.g., a fluoroscope, a CT scanner, or other X-ray machine), a magnetic resonance imaging (MRI) scanner, an ultrasound imaging device, an optical coherence tomography (OCT) scanner, an endoscope, a microscope, an optical camera, a thermographic camera (e.g., an infrared camera), a radar or LIDAR system (which may comprise, for example, a transmitter, a receiver, a processor, and one or more antennae), or any other imaging device 112 suitable for obtaining images of an anatomical feature of a patient. The imaging device 112 may be contained entirely within a single housing, or may comprise a transmitter/emitter and a receiver/detector that are in separate housings or are otherwise physically separated.

In some embodiments, the imaging device 112 may comprise more than one imaging device 112. For example, a first imaging device may provide first image data and/or a first image, and a second imaging device may provide second image data and/or a second image. In still other embodiments, the same imaging device may be used to provide both the first image data and the second image data, and/or any other image data described herein. The imaging device 112 may be operable to generate a stream of image data. For example, the imaging device 112 may be configured to operate with an open shutter, or with a shutter that continuously alternates between open and shut so as to capture successive images. For purposes of the present disclosure, unless specified otherwise, image data may be considered to be continuous and/or provided as an image data stream if the image data represents two or more frames per second. As will be discussed in further detail herein, an imaging device 112 may be mounted to a robot 114 or robotic arm 116 and may be configured to obtain images of tracking markers that are also being imaged by other imaging devices 112 not mounted to the robot 114. In some embodiments, images obtained from the imaging device 112 mounted to the robot 114 may be used to supplement image data at the navigation system 118 obtained from other imaging devices 112. It may also be possible to track a motion of the imaging device 112 that is mounted to the robot 114 to determine a motion and pose of the robot 114 and the imaging device 112 relative to the surgical area.

The robot 114 may be any surgical robot or surgical robotic system. The robot 114 may be or comprise, for example, the Mazor X Stealth Edition robotic guidance system. The robot 114 may be configured to position the imaging device 112 at one or more precise position(s) and orientation(s), and/or to return the imaging device 112 to the same position(s) and orientation(s) at a later point in time. The robot 114 may additionally or alternatively be configured to manipulate a surgical tool (whether based on guidance from the navigation system 118 or not) to accomplish or to assist with a surgical task. In some embodiments, the robot 114 may be configured to hold and/or manipulate an anatomical element during or in connection with a surgical procedure. The robot 114 may comprise one or more robotic arms 116. In some embodiments, the robotic arm 116 may comprise a first robotic arm and a second robotic arm, though the robot 114 may comprise more than two robotic arms. In some embodiments, one or more of the robotic arms 116 may be used to hold and/or maneuver the imaging device 112. In embodiments where the imaging device 112 comprises two or more physically separate components (e.g., a transmitter and receiver), one robotic arm 116 may hold one such component, and another robotic arm 116 may hold another such component. Each robotic arm 116 may be positionable independently of the other robotic arm. The robotic arms may be controlled in a single, shared coordinate space, or in separate coordinate spaces.

The robot 114, together with the robotic arm 116, may have, for example, one, two, three, four, five, six, seven, or more degrees of freedom. Further, the robotic arm 116 may be positioned or positionable in any pose, plane, and/or focal point. The pose includes a position and an orientation. As a result, an imaging device 112, surgical tool, or other object held by the robot 114 (or, more specifically, by the robotic arm 116) may be precisely positionable in one or more needed and specific positions and orientations.

The robotic arm(s) 116 may comprise one or more sensors that enable the processor 104 (or a processor of the robot 114) to determine a precise pose in space of the robotic arm (as well as any object or element held by or secured to the robotic arm).

In some embodiments, reference markers (i.e., navigation markers) may be placed on the robot 114 (including, e.g., on the robotic arm 116), the imaging device 112, or any other object in the surgical space. The reference markers may be tracked by the navigation system 118, and the results of the tracking may be used by the robot 114 and/or by an operator of the system 100 or any component thereof. In some embodiments, the navigation system 118 can be used to track other components of the system (e.g., imaging device 112) and the system can operate without the use of the robot 114 (e.g., with the surgeon manually manipulating the imaging device 112 and/or one or more surgical tools, based on information and/or instructions generated by the navigation system 118, for example).

The navigation system 118 may provide navigation for a surgeon and/or a surgical robot during an operation. The navigation system 118 may be any now-known or future-developed navigation system, including, for example, the Medtronic StealthStation™ S8 surgical navigation system or any successor thereof. The navigation system 118 may include one or more cameras or other sensor(s) for tracking one or more reference markers, navigated trackers, or other objects within the operating room or other room in which some or all of the system 100 is located. The one or more cameras may be optical cameras, infrared cameras, or other cameras. In some embodiments, the navigation system may comprise one or more electromagnetic sensors. In various embodiments, the navigation system 118 may be used to track a position and orientation (e.g., a pose) of the imaging device 112, the robot 114 and/or robotic arm 116, and/or one or more surgical tools (or, more particularly, to track a pose of a navigated tracker attached, directly or indirectly, in fixed relation to the one or more of the foregoing). The navigation system 118 may include a display for displaying one or more images from an external source (e.g., the computing device 102, imaging device 112, or other source) or for displaying an image and/or video stream from the one or more cameras or other sensors of the navigation system 118. In some embodiments, the system 100 can operate without the use of the navigation system 118. The navigation system 118 may be configured to provide guidance to a surgeon or other user of the system 100 or a component thereof, to the robot 114, or to any other element of the system 100 regarding, for example, a pose of one or more anatomical elements, whether or not a tool is in the proper trajectory, and/or how to move a tool into the proper trajectory to carry out a surgical task according to a preoperative or other surgical plan.

The system 100 or similar systems may be used, for example, to carry out one or more aspects of any of the methods 500 and 600 described herein. The system 100 or similar systems may also be used for other purposes. Any of the methods depicted and described herein may be carried out or otherwise performed, for example, by at least one processor. The at least one processor may be the same as or similar to the processor(s) 104 of the computing device 102 described above. The at least one processor may be part of a robot (such as a robot 114) or part of a navigation system (such as a navigation system 118). A processor other than any processor described herein may also be used to execute the methods 500 and/or 600. The at least one processor may perform a method (e.g., method 500 or 600) by executing instructions stored in a memory such as the memory 106.

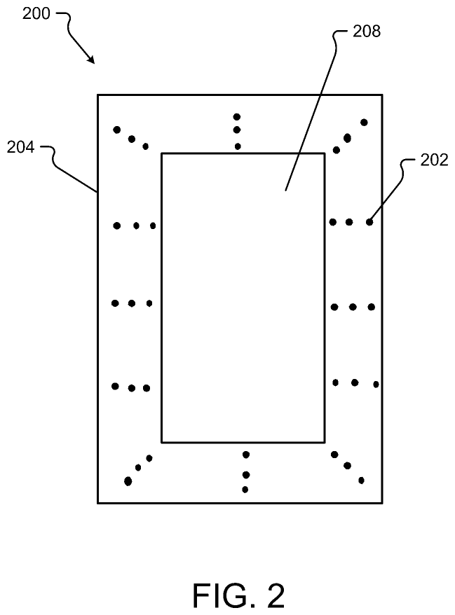

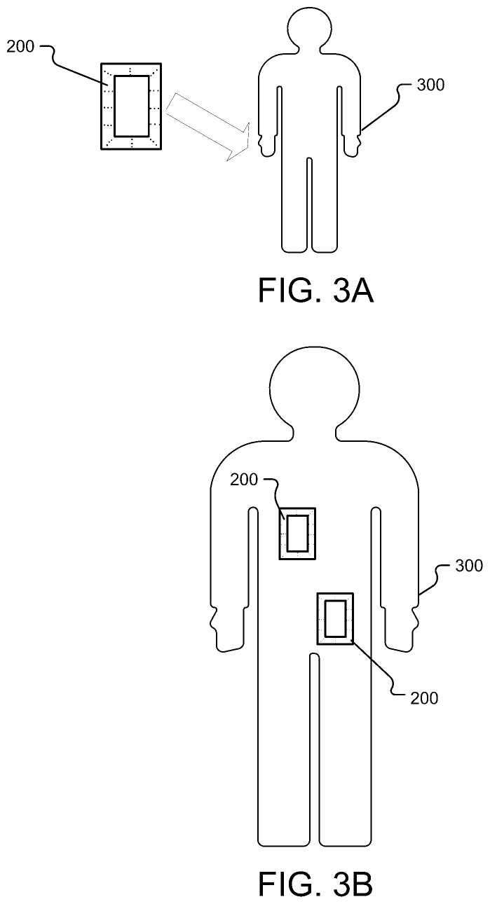

Referring now to FIGS. 2, 3A, and 3B, additional details of fiducials and other navigation markers to be used in tracking soft tissue motion will be described in accordance with at least some embodiments of the present disclosure. One or more tracking markers 202 may be provided on a flexible material 204 and the flexible material 204 may be configured for attachment to an object or patient 300 (e.g., on skin, an organ, or other soft tissue of the patient 300) to assist with the tracking of soft tissue motion intraoperatively and/or postoperatively. In some embodiments, a flexible material 204 may include an array of markers 200 provided thereon, where each tracking marker 202 in the array of markers 200 is distributed in a known pattern across the flexible material 204.

As illustrated in FIG. 2, the tracking markers 202 may be radially disposed relative to a center area 208 of the array 200. In particular, the center area 208 may correspond to a portion of the array 200 in which an incision is made. The center area 208 may or may not have a tracking marker 202 provided thereon. In some embodiments, the tracking markers 202 are positioned around the center area 208 in a pattern that will enable a determination of motion relative to the center area 208. Because the center area 208 may correspond to a location where an incision is made during a surgical procedure, the surrounding tracking markers 202 can be tracked with an imaging device 112 to determine a motion of the soft tissue around the surgical area (e.g., the area inside the center area 208). In some embodiments, the tracking markers 202 are provided on a ribbon that surrounds the center area 208.

In some embodiments, the tracking markers 202 may be integrated into the flexible material 204 or affixed to the flexible material 204 such that motion of the flexible material 204 translates to a motion of the tracking markers 202. More specifically, in one example, the tracking markers 202 may be printed into the flexible material 204 such that the profile of the array 200 is substantially constant across the entirety of the array 200. In other words, the tracking markers 202 may be integrated into the flexible material as printed dots, squares, circles, or the like, which means that motion of the flexible material 204 is immediately translated to motion in a tracking marker 202.

Illustratively, but without limitation, the flexible material 204 may be configured for attachment to a patient 300 or an object of interest (e.g., a soft tissue element belonging to a patient 300 such as an organ, nerve, muscle, tumor growth, skin, bone, etc.). As shown in FIG. 3B, multiple patches of flexible material 204 may be attached to a patient 300 or one or more anatomical elements of a patient 300. With the flexible material 204 attached to the patient 300 or object of a patient 300, the object identification 122 and/or tissue motion tracking 124 of the computing device 102 may be configured to identify and track motion of soft tissue elements belonging to a patient 300 intraoperatively and/or postoperatively. Specifically, but without limitation, because the flexible material 204 may include one or more tracking markers 202, the position and motion of tracking markers 202 can be correlated to a motion of the flexible material 204, which is correlated to a motion of an object to which the flexible material 204 is attached.

The flexible material 204 may be attached or connected to a patient 300 or object of a patient 300 in a number of ways. In some embodiments, attachment mechanisms (e.g., connectors, staples, sutures, glue, etc.) may be used to attach the flexible material 304 to the patient 300 or anatomical element of the patient 300. In some embodiments, the flexible material 204 may be provided with integrated attachment mechanisms, which enable the flexible material 204 to releasably connect with or attach to the patient 300 or anatomical element of the patient 300. The flexible material 204 may correspond to a 3D printed elastic material, rubber, thin paper, a woven material, a non-woven material, or the like. The tracking markers 202 may be created using the same technique used to create the flexible material 204, but a color of the tracking markers 202 may vary from other portions of the flexible material 204 that are not intended to correspond to tracking markers 202.

In some examples, the flexible material 204 may correspond to a mesh or film that is configured to wrap around an object and accept a shape of the object once attached thereto. The flexible material 204 may correspond to a membrane that connects to a soft tissue element of a patient 300. The flexible material 204 may be attachable to a nerve, spinal cord, stent, or blood vessel that should not be cut during a surgical procedure. In this way, the flexible material 204 may be used to track an object that should not be cut or move in response to contact by the robot 114. Alternatively or additionally, the flexible material 204 may be attachable to an object that should be cut during a surgical procedure. Examples of such objects include, without limitation, tumors, growths, organs, foreign objects (e.g., non-human objects), etc. Although the present disclosure is directed primarily to the tracking of soft tissue, embodiments hereof may additionally or alternatively be used to track hard tissue as well, including by attaching a flexible material 204 comprising an array of markers 200 to a bone or other hard tissue of a patient.

The flexible material 204 may be configured for removal from the patient 300 when the surgical procedure is complete or the flexible material 204 may be configured to be left in the patient 300 postoperatively. In some embodiments, the flexible material 204 may include a bioabsorbable mesh that is configured to dissolve after being left in the patient 300 for more than a predetermined amount of time. In some embodiments, the flexible material 204 may include an optically transparent or semitransparent material that does not obstruct a view of an object to which the flexible material 204 is attached. In some embodiments, the flexible material 204 may be configured to be cut during surgery, but the cut flexible material 204 still retains some degree of attachment to the object when the flexible material 204 and object are cut at the same time.

The tracking markers 202 may correspond to active and/or passive fiducials. Tracking markers 202 may vary according to whether they are active or inactive, whether they respond to certain imaging techniques (e.g., visible light, ultrasound, magnetic resonance, radiation, LIDAR, etc.). As an example, some tracking markers 202 may correspond to fluorescent ink, visible ink, heat-activated ink, or the like. In some embodiments The tracking markers 202 may correspond to fiducials that are relatively small in size (e.g., between 2 mm and 4 mm) so that they are visible to the human eye, but do not hinder a visualization of the object to which the flexible material 204 is connected. A space between adjacent tracking markers 202 may be larger than a size of the tracking markers 202, but such a configuration is not required. The size of the tracking markers 202 and/or the size of space between tracking markers 202 may depend upon the resolution of the imaging device 112 used to track the tracking markers 202. For instance, a 3D camera may be connected to a robot 114 and may be used to obtain close-up images of the tracking markers 202 because the 3D camera is closer to the surgical area than other imaging devices 112. In some examples, the camera may have a resolution of 0.1 mm, which means that tracking markers 202 of 1 mm or 2 mm in size and/or spaces between tracking markers 202 of 1 mm or 2 mm in size would be feasible.

Any one of the tracking markers 202 may be identified and registered by the object identification 122 and/or tissue motion tracking 124 of the computing device 102 to help with the identification of objects and tracking of motion of objects during and/or after a surgical procedure. In some embodiments, each of the tracking markers 202 may be specifically identified once attached to the patient 300 or anatomical element of the patient 300. After identification and registration of the tracking markers 202, the tissue motion tracking 124 may be configured to specifically monitor a position and movement of each individual tracking markers 202 during the surgical procedure. The monitoring may occur continuously or periodically during the surgical procedure to track a motion of soft tissue elements and to help ensure that a navigation and/or surgical plan is followed and/or determine whether a navigation and/or surgical plan should be adjusted.

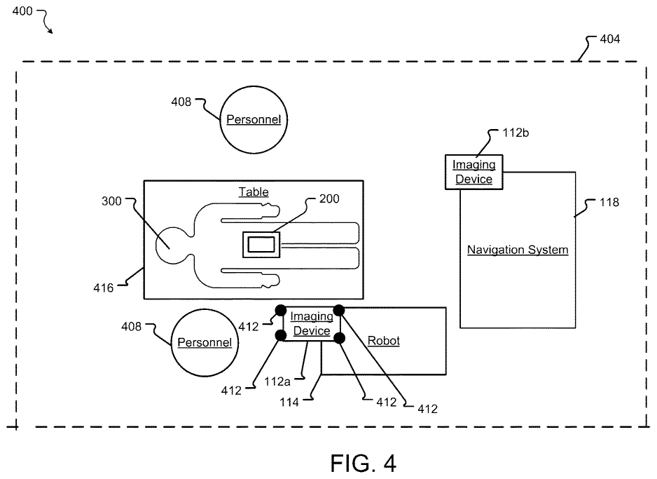

FIG. 4 illustrates additional details of an environment 400 in which the system may be implemented according to at least some embodiments of the present disclosure. The environment 400 in which a robotic system may operate will be described in accordance with at least some embodiments of the present disclosure. The environment 300 may include a surgical environment, such as an operating room. The environment 300 may include a sterile area 404 and a non-sterile area. Objects contained within the sterile area 404 may be considered sterile or “safe” as compared to objects located outside the sterile area 404.

In some embodiments, the patient 300 may be positioned on a table 416 inside the sterile area 316 along with health care personnel 408 (e.g., doctors, surgeons, nurses, support staff, etc.). Some or all of the robot 114 may also be provided within the sterile area 404. A first imaging device 112a may be provided on, held by, or connected to some portion of the robot 114. For instance, the first imaging device 112a may be connected to a robotic arm 116 or end effector of the robot 114. The first imaging device 112a may correspond to a visible light camera. As an example, the first imaging device 112a may correspond to a 3D camera having a resolution of 1 mm per pixel. The first imaging device 112a may be relatively close to a surgical area on which the array of markers 200 are provided. The first imaging device 112a may be configured to have its field of view focused on the patient 300, thereby excluding other objects in the environment 400. As a more specific example, the first imaging device 112a may have its field of view focused on the surgical area around which the array of markers 200 are provided.

The first imaging device 112a is also shown to have multiple tracking markers 412 attached thereto. The tracking markers 412 may correspond to larger fiducials than the tracking markers 202 on the array 200. For instance, the tracking markers may have a size of 10 mm to 100 mm in diameter and may correspond to larger balls or globe-shaped objects that are connected to the first imaging device 112a at one or more predetermined locations.

The navigation system 118 may have access to one or more additional imaging devices 112, such as a second imaging device 112b. The second imaging device 112b may be of the same type as the first imaging device 112a, or it may be of a different type. For instance, the second imaging device 112b may correspond to an ultrasonic imaging device, an infrared imaging device, a magnetic resonance imaging device, a 2D camera, or a 3D camera.

The second imaging device 112b may not be as close to the patient 300 as the first imaging device 112b, but may have a field of view that captures the patient 300 as well as the first imaging device 112b. In this way, images obtained from the second imaging device 112b may be used to determine a position of the first imaging device 112a relative to the patient 300 and to the surgical area. As will be described in further detail herein, the navigation system 118 may be configured to receive image data from the first imaging device 112a and second imaging device 112b to determine motions of the patient 300 (e.g., breathing, heaving, etc.) and to determine positions of the robot 114 relative to the patient 300. Such information can be used to support the robot's 114 position and pose during a surgical procedure.

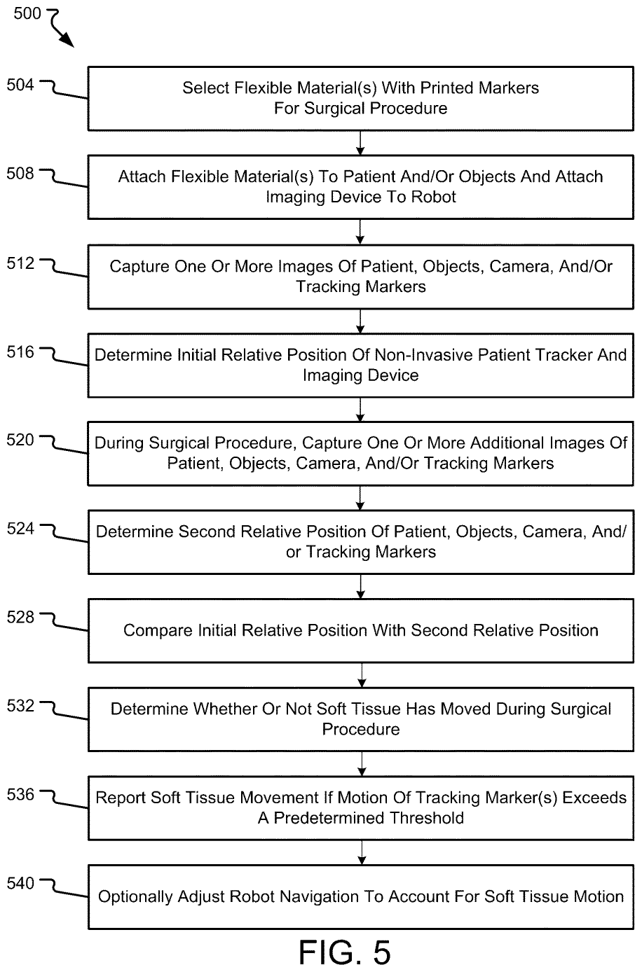

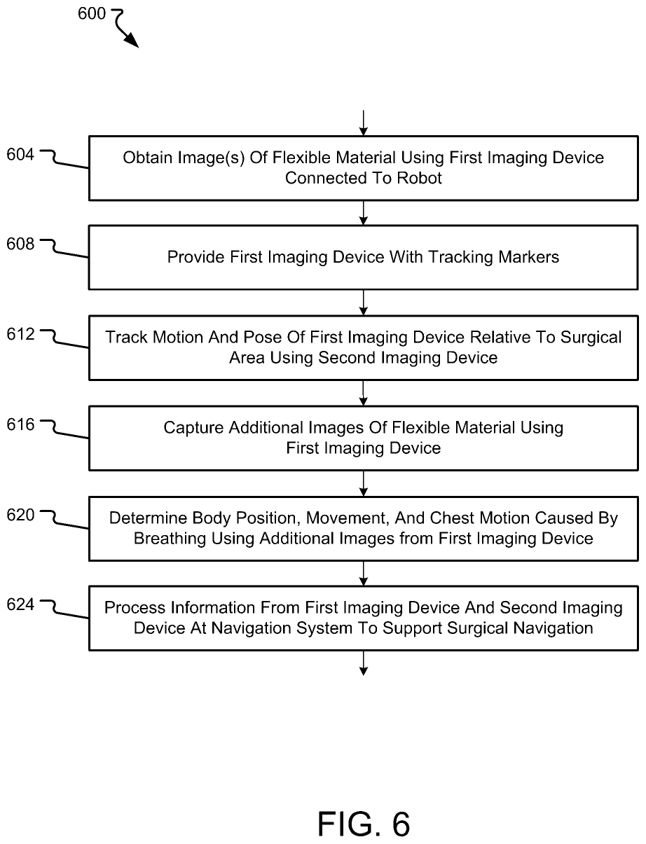

With reference now to FIGS. 5 and 6, various methods will be described in accordance with at least some embodiments of the present disclosure. It should be appreciated that the methods and/or method steps can be performed in any order and may executed by any one or combination of components depicted and described with reference to system 100. Furthermore the steps of one method can be combined, in any order, with steps of any other method without departing from the scope of the present disclosure.

Referring initially to FIG. 5, a surgical method 500 will be described according to at least one embodiment of the present disclosure. The method 500 (and/or one or more steps thereof) may be carried out or otherwise performed, for example, by at least one processor. The at least one processor may be the same as or similar to the processor(s) 104 of the computing device 102 described above. The at least one processor may be part of a robot (such as a robot 114) or part of a navigation system (such as a navigation system 118). A processor other than any processor described herein may also be used to execute the method 500. The at least one processor may perform the method 500 by executing data stored in a memory such as the memory 106. The data (e.g., instructions or neural networks) may cause the processor to execute one or more of the image processing 120, object identification 122, tissue motion tracking 124, and/or reporting and feedback 126.

The method 500 begins by selecting one or more flexible materials 204 for use during a surgical procedure (step 504). The selected flexible materials 204 may be of the same or different types (e.g., mesh, film, membrane, etc.). The selected flexible material(s) 204 may include one or many tracking markers 202 of the same or different types. In some embodiments, the flexible material(s) 204 may be selected based on an object to which the flexible material 204 will be attached. For example, the size, shape, or type of object to which the flexible material 204 will be attached may determine the type of flexible material 204 selected for the object. The flexible material 204 may also be selected based on the type(s) of tracking markers 202 provided thereon and/or capabilities of an imaging device 112 that will track a location of the tracking markers 202.

The method 500 may continue by attaching the flexible material(s) 204 to the patient 300 and/or anatomical element of the patient 300 (step 508). This step may further include attaching an imaging device 112 to a robot 114 that is located in proximity of the surgical area. As can be appreciated, the flexible material(s) 204 may be attached to one or multiple objects and may be configured to conform to the shape of the object(s) once attached thereto. The attachment mechanism(s) used to attach the flexible material(s) 204 to an object may depend upon the expected amount of motion the object will undergo during surgery. For instance, if the object is not expected to move significantly (e.g., less than a few centimeters or millimeters) during surgery, then a first type of attachment mechanism may be used (e.g., nano-hooks) whereas if the object is expected to move significantly (e.g., more than a few centimeters or millimeters) during surgery, then a second type of attachment mechanisms may be used (e.g., staples, sutures, glue, etc.). The flexible material(s) 204 may be attached during a minimally invasive procedure, or during an open procedure. The flexible material(s) 204 may be attached prior to commencing a surgical plan that includes monitoring a pose of soft tissue while one or more other surgical steps are completed. In some embodiments, the flexible material(s) 204 may be attached intraoperatively, but before soft tissue movement monitoring is needed. The imaging device 112 may be attached to the robot 114 in a way that enables the imaging device 112 to capture images of the tracking markers 202. In other words, the imaging device 112 may be connected to the robot 114 such that the imaging device's 112 field of view captures the surgical area and the tracking markers 202 surrounding the surgical area. The imaging device 112 connected to the robot 114 may have one or more tracking markers 412 attached thereto that enable tracking of the imaging device 112 via another imaging device 112.

The method 500 may then continue by capturing one or more images of the patient 300, objects of the patient 300, the imaging device 112 connected to the robot 114, and/or tracking markers 202, 412 (step 512). The images may include any type of image or image data generated with machine vision techniques. Non-limiting examples of imaging techniques that may be used to capture the one or more images include ultrasound imaging, magnetic resonance imaging, fluoroscopic imaging, infrared imaging, visible light imaging, radiation imaging, computed tomography imaging, nuclear medicine imaging, positron-emission tomography, combinations thereof, and the like. The images may include preoperative images, in which case the images may not necessarily include the tracking markers 202, 412 (if, for example, the flexible material(s) 204 have not yet been attached to the patient). The images may include intraoperative images, which may or may not include tracking markers 202, 412. The images may include postoperative images, which may or may not include tracking markers 202, 412. In some embodiments, the images may be received directly from an imaging device 112 or from a computer device that stored an electronic copy of the image. The image(s) received in step 512 may include pixel-based images and/or model-based images. In some embodiments, the step 512 may comprise generating a two-dimensional or three-dimensional model from one or more images captured during the step 406, using known model generation techniques.

Where the image(s) captured in the step 512 do(es) include the tracking markers 202, 412, the method 500 may then continue with the processor 104 executing the object identification 122 and/or tissue motion tracking 124 to determine an initial relative position of the tracking markers 202, 412 (step 516). In particular, image data from the imaging device 112 connected to the robot 114 may identify a position of tracking markers 202 while image data from another imaging device 112 not connected to the robot 114 may identify a position of the tracking markers 412. The combination of image data from these multiple imaging devices 112 may help determine a relative position of the robot 114 and the robotic arm 116 relative to the surgical area and the patient 300. The initial relative position of the tracking markers 202, 412 may be used to determine an initial position (absolute or relative) of one or more objects in the image. The position may be determined relative to other objects, relative to an arbitrary coordinate origin, or combinations thereof.

The method 500 may then include capturing one or more additional images of the patient 300, of the anatomical element of the patient 300, and/or of the tracking markers 202, 412 during the surgical procedure (step 520). The image(s) captured intraoperatively may utilize the same or different type of imaging technique as was used to capture images in step 516. The image(s) may correspond to still images and/or video images. The image(s) may be captured continuously during the surgical procedure or periodically (e.g., according to predetermined intervals). Where the one or more additional images captured in the step 520 include the tracking markers 202, 412, the step 520 may comprise registering the tracking markers 202, 412 as depicted in the one or more additional images to the tracking markers 202, 412 as depicted in the one or more images captured during the step 512.

The method 500 may then continue with the processor 104 executing the object identification 122 and/or tissue motion tracking 124 to determine a second position of the tracking markers 202, 412 (step 524). The second position may correspond to an absolute position in space or a position relative to at least one other object or adjacent tracking markers 202, 412. In some embodiments, a motion of a soft tissue element having a flexible material 204 attached thereto may result in one or more tracking markers 202, 412 moving from a first position to a second position between images. The second position of tracking markers 202, 412 may be compared to previous positions of the same tracking markers 202, 412 and/or to current position(s) of other tracking markers 202, 412 (step 528) to determine whether or not the soft tissue element has moved during the surgical procedure (step 532). The processor 104 may execute the object identification 122 and/or tissue motion tracking 124 to perform steps 528 and/or 532.

If the tissue motion tracking 124 determines that the soft tissue element has moved more than a predetermined threshold, then the tissue motion tracking 124 may invoke the reporting and feedback 126 to report soft tissue movement (step 536). In some embodiments, the soft tissue movement may be reported to a motion controller of the robot 114 and/or to a surgeon operating the robot 114. Accordingly, feedback generated in step 536 may include electronic feedback for a motion controller and/or audible/visible feedback for a human user. In some embodiments, the feedback generated in step 536 may be used to optionally adjust a manual or robotic navigation and/or surgical plan that accounts for the soft tissue motion (step 540). The adjustment to the navigation plan may include adjustments to avoid cutting the soft tissue after movement, ensure cutting the soft tissue after movement, or combinations thereof. The feedback generated in the step 536 may additionally or alternatively comprise a recommended modification to a navigation and/or surgical plan, which recommended modification may be generated automatically.

The present disclosure encompasses embodiments of the method 500 that comprise more or fewer steps than those described above, and/or one or more steps that are different than the steps described above.

Referring now to FIG. 6, additional details of a second method 600 will be described in accordance with at least some embodiments of the present disclosure. Operations of method 600 may be combined with operations of method 500 without departing from the scope of the present disclosure.

The method 600 begins by obtaining image(s) of the flexible material 204 using a first imaging device 112a connected to a robot 114 (step 604). The first imaging device 112a may correspond to a visible light camera. For instance, and without limitation, the first imaging device 112a may include a 3D camera having a resolution of 1 mm per pixel. The first imaging device 112a may be connected to a robotic arm 116, an end effector, or some other component of the robot 114 that is supporting the surgical procedure. By attaching the first imaging device 112a to the robot 114, the first imaging device 112a may be in a position to capture better images of the flexible material 204 than other imaging devices 112 in the operating room because the first imaging device 112a is the closest imaging device 112 to the surgical area.

The method 600 may also include providing the first imaging device 112a with one or more tracking markers 412 (step 608). The tracking markers 412 may be connected to the first imaging device 112a such that motion of the first imaging device 112a translates to motion of the tracking markers 412, which can be tracked by other imaging devices in the operating room. In particular, images obtained from a second imaging device 112b may be used to track a motion and/or pose of the first imaging device 112a (step 612). In some embodiments, the motion and pose of the first imaging device 112a may be tracked relative to the surgical area, the flexible material 204, and the tracking markers 202 provided on/in the flexible material 204.

The method 600 may continue by capturing additional images of the flexible material 204 using the first imaging device 112a (step 616). The additional images captured by the first imaging device 112a may be capable of tracking motion of the tracking markers 202 attached to the flexible material 204 whereas other images captured by other imaging devices 112 may not have the ability or resolution required to track a motion of the tracking markers 202. In particular, the images captured by the first imaging device 112a may be used to determine body position of the patient 300, movement of the patient 300, and/or chest motion of the patient 300 caused by breathing (step 620).

The images from the first imaging device 112a and the second imaging device 112b may be provided to the navigation system 118 for processing (step 624). In particular, the navigation system 118 may be configured to process the image data from the first imaging device 112a and other imaging devices 112 (e.g., the second imaging device 112b) to determine whether motion of the patient 300 and/or other objects should be used to support surgical navigation. For instance, the determinations at step 620 can be used to initiate actions as described in operations 536 and/or 540.

The foregoing is not intended to limit the disclosure to the form or forms disclosed herein. In the foregoing Detailed Description, for example, various features of the disclosure are grouped together in one or more aspects, implementations, and/or configurations for the purpose of streamlining the disclosure. The features of the aspects, implementations, and/or configurations of the disclosure may be combined in alternate aspects, implementations, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, implementation, and/or configuration. Thus, the following claims are hereby incorporated into this Detailed Description, with each claim standing on its own as a separate preferred implementation of the disclosure.

Moreover, though the foregoing has included description of one or more aspects, implementations, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, implementations, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

The phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” “A, B, and/or C,” and “A, B, or C” means A alone, B alone, C alone, A and B together, A and C together, B and C together, or A, B and C together.

The term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

The term “automatic” and variations thereof, as used herein, refers to any process or operation, which is typically continuous or semi-continuous, done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material.”

Aspects of the present disclosure may take the form of an implementation that is entirely hardware, an implementation that is entirely software (including firmware, resident software, micro-code, etc.) or an implementation combining software and hardware aspects that may all generally be referred to herein as a “circuit,” “module,” or “system.” Any combination of one or more computer-readable medium(s) may be utilized. The computer-readable medium may be a computer-readable signal medium or a computer-readable storage medium.

A computer-readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer-readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer-readable storage medium may be any tangible medium that can contain or store a program for use by or in connection with an instruction execution system, apparatus, or device.

A computer-readable signal medium may include a propagated data signal with computer-readable program code embodied therein, for example, in baseband or as part of a carrier wave. Such a propagated signal may take any of a variety of forms, including, but not limited to, electro-magnetic, optical, or any suitable combination thereof. A computer-readable signal medium may be any computer-readable medium that is not a computer-readable storage medium and that can communicate, propagate, or transport a program for use by or in connection with an instruction execution system, apparatus, or device. Program code embodied on a computer-readable medium may be transmitted using any appropriate medium, including, but not limited to, wireless, wireline, optical fiber cable, RF, etc., or any suitable combination of the foregoing.

The terms “determine,” “calculate,” “compute,” and variations thereof, as used herein, are used interchangeably and include any type of methodology, process, mathematical operation or technique.

1. A method, comprising:
obtaining, via a first imaging device, a first image of an object in a surgical area, wherein the first image comprises one or more first tracking markers arranged in a first position relative to the object;
obtaining, via a second imaging device, a second image of the first imaging device, wherein the second image comprises one or more second tracking markers arranged in a first position;
obtaining, via the first imaging device, one or more additional first images of the object, wherein the one or more additional first images comprise the one or more first tracking markers;
obtaining, via the second imaging device, one or more additional second images of the first imaging device, wherein the one or more additional second images comprise the one or more second tracking markers; and
determining, based on the one or more additional first images and the one or more additional second images, whether the object has moved and/or whether the first imaging device has moved relative to the object.

2. The method of claim 1, wherein the object comprises soft tissue, the method further comprising:
determining, based on the one or more additional first images, that the object has moved beyond a predetermined object motion threshold; and
in response to determining that object has moved beyond the predetermined object motion threshold, reporting soft tissue motion to at least one of a surgeon and a robot.

3. The method of claim 2, wherein the soft tissue motion is reported to the robot, the method further comprising:
adjusting a robotic surgical plan based on the soft tissue motion.

4. The method of claim 2, wherein the first imaging device is connected to the robot.

5. The method of claim 4, wherein the first imaging device is connected to at least one of a robotic arm and an end effector of the robot.

6. The method of claim 4, further comprising:
determining, based on the one or more additional second images, that the first image device has moved relative to the object beyond a predetermined distance threshold;
correlating motion of the first imaging device to motion of the robot; and
in response to determining that the first image device has moved relative to the object beyond a predetermined distance threshold, reporting robot or patient movement to the surgeon.

7. The method of claim 1, wherein the one or more first tracking markers are provided on a flexible material and wherein the flexible material is attached to a patient at or near a surgical area.

8. The method of claim 7, wherein the flexible material is attached to the patient using at least one of a staple, suture, and glue.

9. The method of claim 7, wherein the one or more first tracking markers are sized according to a resolution of the first imaging device and are not visible to the second imaging device.

10. The method of claim 7, wherein the one or more first tracking markers are printed on the flexible material.

11. The method of claim 10, wherein the flexible material comprises a center area that is substantially devoid of the one or more first tracking markers and wherein the one or more first tracking markers are disposed radially around the center area.

12. The method of claim 11, wherein the center area comprises an area through which an incision is made.

13. The method of claim 7, wherein the flexible material comprises a transparent or semitransparent material.

14. The method of claim 7, wherein the one or more second tracking markers are larger than the one or more first tracking markers and wherein the one or more second tracking markers are attached to the first imaging device.

15. The method of claim 1, further comprising:
determining, based on the one or more additional first images, that the one or more first tracking markers have moved to a second position relative to the object;
determining, based on the one or more additional second images, that the one or more second tracking markers have moved to a second position indicating a motion of the first imaging device relative to the object; and
reporting motion of the object and motion of the first imaging device.

16. The method of claim 1, wherein at least one of the first image and the second image include one or more of an ultrasound image, a magnetic resonance image, a fluoroscopic image, an infrared image, a visible light image, a radiation image, a computed tomography image, a nuclear medicine image, and a positron-emission tomography image.

17. A system, comprising:
a first imaging device comprising a first field of view that captures one or more first tracking markers arranged on or around a surgical area;
a second imaging device comprising a second field of view that captures the first imaging device; and
a computing device, wherein the computing device comprises:
a processor; and
computer memory coupled with the processor and having data stored therein that, when executed by the processor, enables the processor to:
receive first image data from the first imaging device, wherein the first image data indicates a motion of the one or more first tracking markers relative to the surgical area;
receive second image data from the second imaging device, wherein the second image data indicates a motion of the first imaging device relative to the surgical area; and
determine, based on a combination of the first image data and the second image data, whether an object in the surgical area has moved and/or whether the first imaging device has moved relative to the object.

18. The system of claim 17, wherein one or more second tracking markers are attached to the first imaging device, wherein the one or more first tracking markers are smaller than the one or more second tracking markers, wherein the second field of view includes the surgical area, and wherein the one or more first tracking markers are too small to be recognized in the second image data.

19. The system of claim 17, wherein the first imaging device is connected to a robot that is supporting a surgical procedure at the surgical area and wherein the first imaging device comprises a 3D camera.

20. A surgical system, comprising:
a robot configured to execute a surgical plan;
a first imaging device attached to the robot and configured to capture images of one or more first tracking markers arranged on or around a surgical area, wherein the first imaging device comprises one or more second tracking markers attached thereto;
a second imaging device configured to capture images of the first imaging device and the one or more second tracking markers attached thereto; and
a computing device comprising:
a processor; and
computer memory coupled with the processor and having data stored therein that, when executed by the processor, enables the processor to:
receive first image data from the first imaging device, wherein the first image data indicates a motion of the one or more first tracking markers relative to the surgical area;
receive second image data from the second imaging device, wherein the second image data indicates a motion of the first imaging device relative to the surgical area;
determine, based on a combination of the first image data and the second image data, that an object in the surgical area has moved and/or the first imaging device has moved relative to the object; and
in response to determining that the object in the surgical area has moved and/or the first imaging device has moved relative to the object, update the surgical plan.

21-37. (canceled)
